(19) United States
(12) Patent Application Publication
(10) Pub. No.: US 2026/0157353 A1
(43) Pub. Date: Jun. 11, 2026

(54) LED LIGHT SOURCE FOR ADULT HERMETIA ILLUCENS BREEDING AND ADULT HERMETIA ILLUCENS BREEDING METHOD

(71) Applicant: JIANGSU ACADEMY OF AGRICULTURAL SCIENCES, Jiangsu (CN)

(72) Inventors: Ting HAN, Jiangsu (CN); Xiaomei YE, Jiangsu (CN); Cong WANG, Jiangsu (CN); JING DU, Jiangsu (CN); XIAOTAN HAN, Jiangsu (CN)

(21) Appl. No.: 18/705,209

(22) PCT Filed: Jul. 6, 2022
(86) PCT No.: PCT/CN2022/104132

§ 371 Date: Apr. 26, 2024

(30) Foreign Application Priority Data
Jun. 28, 2022 (CN) 202210747476.6

(51) Int. Cl. A01K 67/36

(52) U.S. Cl. CPC A01K 67/36

(57) ABSTRACT

An LED light source for adult Hermetia illucens breeding and an adult Hermetia illucens breeding method. The LED light source for factory breeding of adult Hermetia illucens comprises four wavebands of light, respectively having the following wavelengths and proportions: 360-370 nm of ultraviolet light accounting for 4-10%; 440-460 nm of blue light accounting for 41-50%; 510-520 nm of green light accounting for 32-39%; and 630-650 nm of red light accounting for 8-15%. The LED light source can shorten the adult breeding cycle, thereby greatly improving the spawning production efficiency.

### TECHNICAL FIELD

The present invention belongs to the field of insect farming, and relates to the farming of the Diptera insect black soldier flies, in particular to an LED light source for promoting mating and spawning of adult black soldier flies during industrialized farming of black soldier flies.

### BACKGROUND ART

Black soldier flies, the scientific name of which is Hermetia illucens L., belong to Diptera order, Stratiomyidae family, Hermetia genus, are widely distributed in the regions between 45° north latitude and 40° south latitude, and are a kind of saprophagous environmental resource insects. A metamorphosis generation of black soldier flies can be completed in 30 to 40 days through five stages: egg, larva, prepupa, pupa and adult, wherein, the black soldier flies eat a lot and store nutrients only in the larva stage, and can transform organic solid wastes such as kitchen waste, food processing waste and livestock manure, etc. The larval body of black soldier flies has a protein content of more than 40% and is also rich in unsaturated fatty acids such as lauric acid, so it is a potential source of feed and bait protein, and has been listed by Food and Agriculture Organization of the United Nations as one of the most important inserts for substituting protein feed in the future.

A stable egg supply technique is the most difficult point in the large-scale farming of black soldier flies, and the light environment for adult breeding is the key factor for stable egg supply. After the emergence, adult black soldier flies mate in 1-2 days under light, then female black soldier flies lay eggs in 1-2 days after the mating. If adult black soldier flies receive no appropriate light irradiation within the window period of 5 days after the emergence, they will lose their reproductive ability. The light environment factors that have influence on the mating and spawning of adult black soldier flies are mainly light quality (light spectrum) and light intensity. Based on the adaptive inheritance of the species to the environment through long-term natural evolution, the best breeding effect of adult black soldier flies is achieved under sunlight irradiation. Although at present the common plastic greenhouse breeding employs outdoor sunlight as the light source, the breeding mode is extensive, the light environment is seriously limited by factors such as season, weather and geographic location. Therefore, the amount of eggs is extremely unstable and the commercial output value is very low. Industrialized farming is the current main developing trend in the black soldier fly farming industry because it can provide a stable and controllable farming environment and has a high automation level. In industrialized farming, the natural light source can be fully replaced by artificial light source, thereby the natural rhythm obstacle can be overcome, and stable annual egg supply can be achieved.

Some researchers attempted to use artificial light sources close to sunlight in full spectrum or partial spectrum to stimulate adult black soldier flies to mate and lay eggs, but failed to achieve an ideal result. For example, Zhang et al. (2010) employed a 500 W iodine-tungsten lamp to irradiate black soldier flies, but only achieved a breeding effect equivalent to 60% of the breeding effect under sunlight; Heusslur et al. (2018) used a monochromatic LED, a fluorescent lamp and a iodine-tungsten lamp to irradiate black soldier flies, but found that there was no significant difference among the effects of the three in promoting egg production of black soldier flies, and none of them could fully replace sunlight; Liu et al. (2020) used a iodine-tungsten lamp, a metal halide lamp, a white-ultraviolet LED and a mixed-color LED, and found that the first three lamps were inferior to the mixed-color LED in stimulating the breeding of black soldier flies, but didn't give an exact light quality ratio of the mixed-color LED, and didn't mention the result of comparison between mixed-color LED and sunlight.

Among the above artificial light sources, the iodine-tungsten lamp and the metal halide lamp have high energy consumption, generate heat greatly and have poor safety for phototactic insects, while the fluorescent lamp has a short life and poor stability, and has a stroboscopic effect that is bad for insect farming. LEDs have low energy consumption, low heat generation, high controllability and customizable wavelength, and are ideal lamps that can fully replace sunlight in the industrialized breeding of black soldier flies.

The Patent No. CN107810927A only described that 530-560 nm green light and 580-620 nm yellow light, which are used in combination at a ratio of 1:1 to irradiate black soldier flies, achieved a better effect in egg amount and fertilization ratio than the situation that they were used separately to irradiate black soldier flies, but didn't involve light in other bands and didn't provide any specific description on the optimization of the light quality ratio; the Patent No. CN108012996A mentioned that the light in any other band was ineffective for black soldier flies except 355-380 nm ultraviolet light, 420-470 nm blue light and 500-535 nm green light, but also didn't provide any specific description on the optimization of the light quality ratio; the Patent No. CN110199959A mentioned that seven LED beads, i.e., ultraviolet, blue, green, red, yellow, violet, orange and cyan LED beads, were used to simulate the solar spectrum and presented the ratio of the beads, but didn't mention the respective band range of various light sources; in fact, the light with wavelength greater than 650 nm in the solar spectrum is physiologically ineffective for black soldier flies; the Patent No. CN108386731A described that LED light combined from 390-435 nm violet light, 492-577 nm green light, 435-450 nm blue light and 2,700-3,200 K warm white light was better than sunlight and iodine-tungsten lamp in stimulating the mating and spawning of black soldier flies, but didn't mention the function of ultraviolet light with a wavelength of below 400 nm, and didn't present the specific spectral range of the warm white light.

### SUMMARY OF THE INVENTION

The technical problem to be solved by the present invention is to provide an LED light source and a method for industrialized breeding of adult black soldier flies, which can achieve stable spawning and high spawning rate.

To solve the technical problem described above, the present invention employs the following technical solution:

An LED light source for industrialized breeding of adult black soldier flies, wherein, said LED source comprises four bands of light, and the wavelength and proportion of the light in each band are as follows respectively:
360-370 nm ultraviolet light, accounting for 4-10%;
440-460 nm blue light, accounting for 41-50%;
510-520 nm green light, accounting for 32-39%;
and 630-650 nm red light, accounting for 8-15%.

In the LED light source of the present invention, the light quality proportion of each light is calculated according to the percentage of the light intensity of the light in a band with respect to the total light intensity, for example, if the total light intensity of the combined light is 120 μmol/m²/s and the light intensity of 440-460 nm blue light is 58 μmol/m²/s, then the ratio of the blue light is 48.33%.

The blue light accounts for 44-46%; and the green light accounts for 34-36%.

The ratio of the ultraviolet light to the blue light to the green light to the red light is 1:5:4:1 or 1:10:8:3.

The total light intensity of the LED light source is 80-200 μmol/m²/s. If the total light intensity of the light source is within the range of 80-200 μmol/m²/s, a good stimulation effect on the breeding of adult black soldier flies can be achieved. If the light intensity is lower or higher than that range, the egg amount of black soldier flies will be decreased significantly. The total light intensity for irradiation may be increased appropriately according to the actual light source layout in the breeding room, so that the average light intensity received actually in the main habitat and flight space of the adult black soldier flies is within the above-mentioned range.

In the present invention, various bands of light of the light source may be in the form of a plurality of separate LED tubes evenly distributed and combined in parallel or maybe in the form of different chip beads which are integrated in one lamp. In order to facilitate manufacturing of the lamp, an ultraviolet LED lamp may be arranged separately from the other three bands of light, but the light spectrum and intensity of the ultraviolet lamp must meet the above-mentioned wavelength and ratio. The power supply for the light source may be one or more DC power sources, the power of which can match the total power of all the lamp tubes (beads).

Compared with the prior art, the present invention attains the following beneficial effects:

The LED light source in the present invention is composed of four kinds of light, i.e., ultraviolet light, blue light, green light and red light, and the ratio of the four kinds of light is specific. The composition and ratio of the light are put forward by the inventor based on long-term research. It is found through experimental study that LEDs with different wavelengths and light quality ratios have great differences in stimulating the breeding of adult black soldier flies. The phytochrome contained in the retinas of a pair of compound eyes and three simple eyes of black soldier flies is perceptive to the light within the range of 300-650 nm, and the light sensitivity peaks appear in the ultraviolet light band, the blue light band and the green light band respectively. Therefore, the inventor selected LEDs with different wavelengths in the range of 300 to 650 nm for a lot of tests with monochromatic light and combined light and so on, and found that the change of light quality components and proportions had a significant influence on the egg amount of adult black soldier flies. When the lights in four bands, i.e., 360-370 nm ultraviolet light, 440-460 nm blue light, 510-520 nm green light and 630-650 nm red light, exist at the same time at a specific ratio, an optimal stimulation effect on the mating and spawning of black soldier flies can be achieved. In addition, each of the lights in the four bands is indispensable to achieve a spawning effect equivalent to or better than the spawning effect under sunlight in industrialized farming so as to totally replace natural light. In the technical solution of the present invention, the 360-370 nm ultraviolet light accounts for 4-10%, the 440-460 nm blue light accounts for 41-50%, the 510-520 nm green light accounts for 32-39%, and the 630-650 nm red light accounts for 8-15%; thus, the mating and spawning effect of black soldier flies is greatly improved.

The brightness of the light combined from blue light, green light and red light in the LED light source in the present invention can meet the perceptual need of human eyes. Therefore, it is unnecessary to provide any other manually operated lighting source in the breeding room equipped with the light source in the present invention. Since the 360-370 nm ultraviolet light in the LED light source of the present invention accounts for only 4-10%, the harm to the health of the operators is negligible, and the operators may take appropriate sun-screening measure and enter the breeding room for work.

The LED light source in the present invention can fully replace the sunlight and is used for indoor industrialized farming of black soldier flies, or may be used as a supplementary light source in the farming facility with light transmission conditions to supplement light in rainy days or winter when the sunlight is inadequate.

The light source having a light cycle within a range of 12 L (light)/12 D (dark) to 16 L/8 D can significantly stimulate the mating and spawning. For example, the light source may be switched on at 7 am and switched off at 9 pm, then the light cycle is 14 L/10 D. Preferably, a timer may be provided at the DC power source to realize automatic control of the light cycle.

The light source may be switched on for irradiation after each batch of pupae of black soldier flies emerge into adults in quantity, and a strong mating-in-pairs behavior can be found after 1-2 days of irradiation, and the daily mating time under the LED light is more ample than the natural rhythm. For example, the mating-in-pairs of black soldier flies mostly occurs between 10 am and 3 pm under sunlight, while the mating behavior can be found at 8 am or 5 pm under the LED light source of the present invention. If a production mode of continuous pupae feeding in batches in different growth periods is employed, it is necessary to shade the pupae to avoid the disturbance of the light source on the emergence process of pupa.

Thanks to the luminescent principle of the LEDs, the energy consumption and generated heat of the light source in the present invention are much lower than those of artificial light sources such as iodine-tungsten lamps and metal halide lamps. Therefore, the farming cost can be reduced in the industrialized breeding process of black soldier flies, and the risk of fire or adult black soldier flies being scalded by the lamp can be greatly decreased. Under the irradiation of the light source, the egg amount, fertilization ratio and other breeding indicators of black soldier flies are significantly better than those under a iodine-tungsten lamp, metal halide lamp, monochromatic LED or sunlight, the adult breeding cycle can be shortened, and the spawning rate can be greatly improved.

### BRIEF DESCRIPTION OF DRAWINGS

FIG. 1 shows the spawning of adult black soldier flies under LED light sources with different single wavelengths;

FIG. 2 shows the spawning of adult black soldier flies under LED light sources with different wavelengths ratios.

### EMBODIMENT

### Example 1: Effects of LED Light Sources with Different Single Wavelengths on the Breeding of Adult Black Soldier Flies

In the experiment, 320 mm×320 mm×320 mm insect breeding cages are used, each cage is filled with 50 male and 50 female adult black soldier flies which pupated in the same batch, and have just emerged in a dark environment within 24 hours. The LED light source with corresponding wavelength is turned on immediately, and the irradiation time is from 7:00 am to 9:00 pm every day, the light cycle is 14 L/10 D, and the light intensity is set to 100 μmol/m²/s. The light source irradiates light on the breeding cage at 90 degrees, and the distance from the light source to the top of the cage is 15 cm. In this experiment, the effects of LED light sources with 8 wavelengths, including ultraviolet light at 290 nm, 330 nm and 365 nm, blue light at 450 nm, green light at 515 nm and red light at 650 nm, 680 nm and 710 nm, on the breeding of adult black soldier flies are studied. Three replicates are used for each group, and altogether 24 breeding cages are used. The LED light sources are LED tubes with a single wavelength.

The temperature of the breeding environment is 28±1° C. and the relative humidity of the breeding environment is 70±5%. Further preferably, water is sprayed into the breeding cages for 3-5 times every day in addition to maintaining the relative humidity of the breeding environment, to ensure that the adult black soldier flies can take sufficient water. 50% decomposed wheat bran and 50% corn flour (mass ratio) are used as the information substance for spawning induction, a spawning plate is placed at the information substance, and the eggs are collected and weighed every day until all groups stops spawning. In this example, the whole breeding cycle of black soldier flies is 8 days.

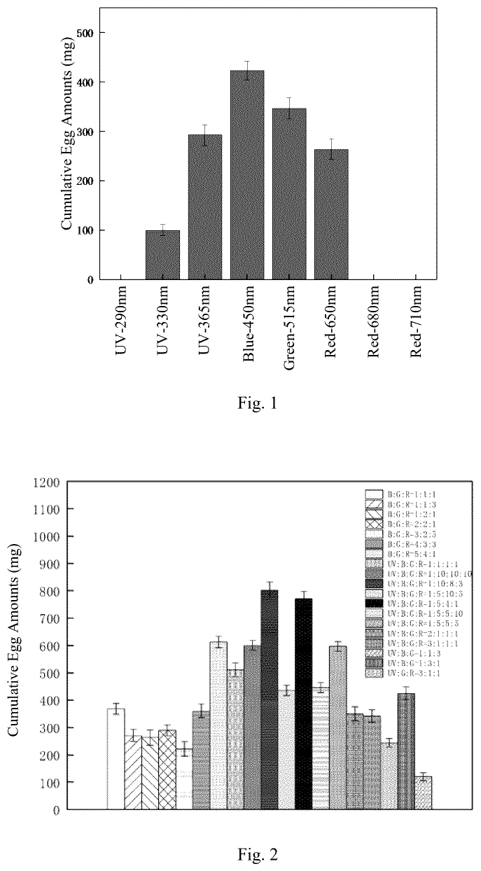

The result is shown in FIG. 1. Among the eight wavelengths, five groups of LED light with wavelengths of 330 nm, 365 nm, 450 nm, 515 nm and 650 nm can stimulate the mating and spawning of the adult black soldier flies, and the cumulative egg amounts in the entire spawning period are 100.00±11.10 mg, 292.33±21.58 mg, 422.67±18.95 mg, 346.00±21.28 mg, and 263.67±21.05 mg respectively; while the other three kinds of light with the wavelengths of 290 nm, 680 nm, and 710 nm fails to stimulate the breeding of the black soldier flies because they are beyond the perception range of the vision system of black soldier flies; therefore the cumulative egg amounts are 0 mg respectively.

This example proves: in the wavelength range of 300-650 nm that can be perceived by the vision system of adult black soldier flies, ultraviolet light, blue light, green light and red light LEDs that have a single wavelength can stimulate the mating and spawning of adult black soldier flies separately, but the stimulation effects of LED light sources with different wavelengths are different from each other. However, the light with 290 nm, 680 nm or 710 nm wavelength can't promote the mating and spawning since it is beyond the perception range of the vision system of black soldier flies.

### Example 2: Effects of LED Light Sources with Different Wavelengths Ratios on the Breeding of Adult Black Soldier Flies

In the experiment, 320 mm×320 mm×320 mm insect breeding cages are used, each cage is filled with 50 male and 50 female adult black soldier flies which pupated in the same batch, and have just emerged in a dark environment within 24 hours. The LED light source with corresponding wavelength is turned on immediately, and the irradiation time is from 7:00 am to 9:00 pm every day, the light cycle is 14 L/10 D.

The effects of LED light sources with four bands of light (365 nm ultraviolet light, 450 nm blue light, 515 nm green light and 650 nm red light) at different ratios, on the breeding of adult black soldier flies are studied. Seven different ratios are studied for the combinations of 450 nm blue light, 515 nm green light and 650 nm red light, which are respectively as following: blue (B):green (G):red (R)=1:1:1, 5:4:1, 1:2:1, 2:2:1, 4:3:3, 1:1:3 and 3:2:5; three different ratios are studied for the combinations of 365 nm ultraviolet light, 450 nm blue light and 515 nm green light, which are respectively as following: ultraviolet (UV):blue (B):green (G)=1:1:3, 1:3:1 and 3:1:1; and nine different ratios are studied for the combinations of 365 nm ultraviolet light, 450 nm blue light, 515 nm green light and 650 nm red light, which are respectively as following: ultraviolet (UV):blue (B):green (G):red (R)=1:1:1:1, 1:10:10:10, 1:10:8:3, 1:5:10:5, 1:5:4:1, 1:5:5:10, 1:5:5:5, 2:1:1:1 and 3:1:1:1. The light intensity of each band is respectively adjusted by means of a sliding rheostat so that the total light intensity is 100 μmol/m²/s. The light source irradiates light on the breeding cage at 90 degrees, and the distance from the light source to the top of the cage is 15 cm. The LED light sources employs single-wavelength LED tubes that are evenly distributed and combined in parallel according to the above ratios. 3 replicates are used for each ratio, and altogether 39 breeding cages are provided for the above 13 wavelength ratios.

The temperature of the breeding environment is 28±1° C. and the relative humidity of the breeding environment is 70±5%. Further preferably, water is sprayed into the breeding cages for 3-5 times every day in addition to maintaining the relative humidity of the breeding environment, to ensure that the adult black soldier flies can take sufficient water. 50% decomposed wheat bran and 50% corn flour (mass ratio) are used as the information substance for spawning induction, a spawning plate is placed at the information substance, and the eggs are collected and weighed every day until all groups stops spawning. In this example, the whole breeding cycle of black soldier flies is 7 days.

The result is shown in FIG. 2. All of the 19 wavelength ratios can stimulate the mating and spawning of the black soldier flies, but there are significant differences in the promotion effect. When the 365 nm ultraviolet light, 450 nm blue light, 515 nm green light and 650 nm red light coexist and are combined at a ratio of 1:5:4:1 or 1:10:8:3, i.e., the blue light and the green light account for about 45% and 36% respectively, the spawning promotion effect is the best, and the cumulative egg amounts in 7 days are 770.67±26.03 mg and 802.4±29.06 mg respectively, which are much higher than those achieved by other wavelength ratios; in addition, there is no significant difference between the two groups.

This example proves: the combination in different ratios of ultraviolet light, blue light, green light and red light, which are within the vision perception range (300-650 nm) of adult black soldier flies, have significantly different influences on the mating and spawning of black soldier flies, the four kinds of light can ensure a high breeding promotion effect when they coexist at an appropriate ratio, and the effect of red light cannot be ignored.

### Example 3: Promotion Effects of Different Light Sources on the Breeding of Adult Black Soldier Flies

The experiment is carried out in four breeding rooms with the same length, width and height (3 m), and with the same area of 6 m². The breeding room 1# has a transparent glass ceiling and the light source is sunlight; the breeding room 2# is equipped with four 500 W iodine-tungsten lamps; the breeding room 3# is equipped with six 400 W metal halide lamps (sodium lamps); the breeding room 4# is equipped with the combined LED in the present invention, which contains four types of beads (i.e., 365 nm ultraviolet light, 450 nm blue light, 515 nm green light and 650 nm red light beads) uniformly mixed at a ratio of 1:5:4:1 in rectangular lamp panels, and the number of the rectangular lamp panels is six. The lamps are arranged evenly so that the average light intensity in the breeding rooms 2#-4# with artificial light sources is 150 μmol/m²/s (measured with a light quantum flux density probe at a position which is 50 cm from the light source), the average light intensity in the breeding room 1# that employs sunlight is 90-250 μmol/m²/s (measured with a light quantum flux density probe at a position which is 45 cm distance from the glass ceiling, with the measurement being performed at a plurality of points and the average being taken), from 6:00 am to 6:00 pm during test.

4 Kg black soldier fly pupae (black, inactive) which are in the same batch and will emerge within 1-2 days are put into each of the four breeding rooms. When the percentage of emergence reaches 80%, the light source is turned on or the light shielding cover is opened (sunlight group 1#), and the light is turned on at 6:00 am and turned off at 6:00 pm every day in the breeding rooms 2-4# that employs artificial light sources, and the light cycle is 12 L/12 D. The temperature of the breeding environment is 28±1° C. and the relative humidity of the breeding environment is 70±5%, 50% decomposed wheat bran and 50% corn flour (mass ratio) are used as the information substance for spawning induction, 10 spawning plates are placed evenly at the information substance on the floor of each breeding room, and the eggs are collected and weighed every day until all groups stops spawning. The collected eggs are placed in a thermo-hygrostat with a temperature of 32° C. and 80±5% relative humidity. After 48 hours, 0.5 g eggs are taken from each treatment group and placed under a microscope (the magnification is 40 times) to observe the hatching situation, and the hatching ratios for different treatment are recorded. Green fake leaf clusters of the same quantity, length and position are placed in each breeding room for the adult black soldier flies to inhabit, and water is sprayed evenly on the fake leaves with a tap water sprayer once every 2 hours for the adult black soldier flies to take water. The result is shown in Table 1.

TABLE 1
Promotion Effects of Different Light Sources on the Breeding of Adult Black Soldier Flies

| | | Pre- | | Cumulative | | | |
|---|---|---|---|---|---|---|---|
| | Light | spawning | Spawning | egg | Fertilization | | Energy |
| Type of | intensity | period | period | amounts | ratio | Power | consumption |
| light source | (μmol/m²/s) | (d) | (d) | (g) | (%) | (w) | (kw · h) |
| Sunlight | 90-250 | 3 | 8 | 135.72 | 92 | — | — |
| Iodine-Tungsten | 150 | 3 | 7 | 89.88 | 88 | 500 × 4 | 240.00 |
| lamp | | | | | | | |
| Metal halide | 150 | 4 | 6 | 72.33 | 85 | 400 × 6 | 288.00 |
| lamp (sodium | | | | | | | |
| lamp) | | | | | | | |
| Combined LED | 150 | 2 | 7 | 169.18 | 96 | 212 × 6 | 137.38 |

The pre-spawning period (the duration from the time the light source is turned on to the time the spawning is first found) for the group of combined LED is only 2 days, and the whole breeding cycle is 9 days, and both times are shorter than those for groups of sunlight, iodine-tungsten lamp and metal halide lamp. The cumulative egg amount of this group is 169.18 g, which is higher than those of the groups of sunlight, iodine-tungsten lamp and metal halide lamp by 24.65%, 88.23% and 133.90% respectively. In addition, the fertilization ratio for the group of combined LED is as high as 96%, which is the highest among the investigated light sources. In the whole breeding period, the energy consumption of the group of combined LED is the lowest, only 137.38 kW·h, which is 57.24% and 47.70% of the energy consumption of the group of iodine-tungsten lamp and the group of metal halide lamp respectively.

This example proves that the LED light source in the present invention is superior to sunlight and the commercially available iodine-tungsten lamps and metal halide lamps commonly used for breeding adult black soldier flies in terms of promoting the breeding of adult black soldier flies, and its energy consumption is much lower than those of iodine-tungsten lamps and metal halide lamps. In this example, the egg amount per unit pupae is 42.30 g/Kg, and the average daily egg amount per unit pupae (based on a 7-day egg period) is 6.04 g/kg/d. In the example of the LED light source for promoting the mating of black soldier flies described in the invention patent No. CN108386731A, the highest average daily egg amount per unit pupae is 5.24 g/kg/d; in the example of the combined LED light device used for breeding black soldier flies described in the invention patent No. CN110199959A, the highest daily egg amount per unit pupae is 3.89 g/Kg; in the example of the LED lighting system for promoting the spawning of adult black soldier flies described in the invention patent No. CN113785798A, the highest egg amount per unit pupae is 21.01 g/kg. Thus, the promotion effect of the LED light source of the present invention on the breeding of black soldier flies is superior to those of the above-mentioned existing technologies.

The LED light source in the present invention also has the functions of shortening the breeding period of adult black soldier flies and improving the efficiency of stable egg supply. It can completely replace sunlight in the feeding process of black soldier flies, break through the limits of season and weather, and make it possible to provide eggs stably throughout the year. In addition, owing to the advantages of low energy consumption, high customization, and convenient automatic control of LEDs, the LED light source is more suitable for large-scale industrialized black soldier fly breeding, and can significantly increase economic benefits for the farming enterprises.

1. An LED light source for breeding adult black soldier flies, wherein, said LED light source comprises four bands of light, the wavelengths and proportions of each band of light are respectively as follows:
360-370 nm ultraviolet light, accounting for 4-10%;
440-460 nm blue light, accounting for 41-50%;
510-520 nm green light, accounting for 32-39%;
and 630-650 nm red light, accounting for 8-15%.

2. The LED light source according to claim 1, wherein the blue light accounts for 44-46%; and the green light accounts for 34-36%.

3. The LED light source according to claim 2, wherein the ratio of the ultraviolet light to the blue light to the green light to the red light is 1:5:4:1.

4. The LED light source according to claim 2, wherein the ratio of the ultraviolet light to the blue light to the green light to the red light is 1:10:8:3.

5. The LED light source according to claim 1, wherein the total light intensity of the LED light source is 80-200 μmol/m²/s.

6. The LED light source according to claim 5, wherein parallel combination of several separate LED tubes is used for various bands of light of the LED light source.

7. The LED light source according to claim 5, wherein different chip beads which are integrated in one lamp is used for various bands of light of the LED light source.

8. The LED light source according to claim 5, wherein an ultraviolet LED lamp and a combined LED lamp having the other three bands of light are used for various bands of light of the LED light source, and the ultraviolet LED lamp is separated from the combined lamp.

9. A method for breeding adult black soldier flies, wherein the LED light source according to claim 1 is used for irradiation.

10. The method for breeding adult black soldier flies according to claim 6, wherein the irradiation method is as follows:
the irradiation time is from 7:00 am to 9:00 pm every day, and the light cycle is 14 L/10 D.
